(19) United States
(12) Patent Application Publication
(10) Pub. No.: US 2026/0047573 A1
(43) Pub. Date: Feb. 19, 2026

(54) NANOSTRUCTURE, METHOD OF MANUFACTURING NANOSTRUCTURE, FILM, AND STRUCTURE INCLUDING FILM

(71) Applicant: SONY GROUP CORPORATION, TOKYO (JP)

(72) Inventor: TAIZO NISHIMURA, TOKYO (JP)

(21) Appl. No.: 19/101,686

(22) PCT Filed: Aug. 3, 2023
(86) PCT No.: PCT/JP2023/028394

§ 371 Date: Feb. 6, 2025

(30) Foreign Application Priority Data
Aug. 16, 2022 (JP) 2022-129643

(51) Int. Cl. A01N 25/34; A01N 37/06; B82Y 30/00

(52) U.S. Cl. CPC A01N 25/34; A01N 37/06; B82Y 30/00

(57) ABSTRACT

A nanostructure according to an embodiment of the present disclosure includes a rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

### TECHNICAL FIELD

The present disclosure relates to a nanostructure having a microstructure on its surface, a method of manufacturing the nanostructure, a film, and a structure including the film.

### BACKGROUND ART

For example, PTL 1 discloses an anti-glare hard coat film improved in a wiping property of an attached fingerprint by including, on one surface of a base, an anti-glare hard coat layer of which a surface has an oleic acid contact angle of 45° or greater.

### CITATION LIST

### Patent Literature

PTL 1: Japanese Unexamined Patent Application Publication No. 2014-232277

### SUMMARY OF THE INVENTION

Incidentally, a hard coat film is desired to have highly durable antibacterial performance.

It is desirable to provide a nanostructure having durable antibacterial performance, a method of manufacturing the nanostructure, a film having durable antibacterial performance, and a structure including the film.

A nanostructure according to an embodiment of the present disclosure includes a rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

A method of manufacturing a nanostructure according to an embodiment of the present disclosure includes forming a random uneven surface in the nanostructure through wet blasting, the random uneven surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

A film according to an embodiment of the present disclosure includes a layer including a nanostructure, the nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

In a structure including a film according to an embodiment of the present disclosure, the film is the film according to the above-described embodiment of the present disclosure.

In the nanostructure according to the embodiment of the present disclosure, the method of manufacturing the nanostructure according to the embodiment of the present disclosure, the film according to the embodiment of the present disclosure, and the structure including the film according to the embodiment of the present disclosure, the random rough surface is provided having the arithmetic mean height (Sa) of 0.09 μm or greater and 1.21 μm or less and the ratio (Sq/Sa) between the root mean square height (Sq) and the arithmetic mean height (Sa) of 1.22 or greater and 2.36 or less. This physically traps and kills bacteria.

### BRIEF DESCRIPTION OF THE DRAWINGS

FIG. 1 is a schematic cross-sectional view of a configuration of a film according to an embodiment of the present disclosure.

FIG. 2 is a schematic explanatory diagram of wettability between trioleic acid and the film illustrated in FIG. 1.

FIG. 3 is a schematic diagram illustrating a vicinity of an interface between the trioleic acid and the film illustrated in FIG. 1.

FIG. 4A is an explanatory diagram of a mechanism of an assumed bactericidal function of the film illustrated in FIG. 1.

FIG. 4B is an explanatory diagram following FIG. 4A of the mechanism of the assumed bactericidal function.

FIG. 4C is an explanatory diagram following FIG. 4B of the mechanism of the assumed bactericidal function.

FIG. 5 is a schematic diagram illustrating Application Example 1 of the present disclosure.

FIG. 6 is a schematic diagram illustrating Application Example 2 of the present disclosure.

### MODES FOR CARRYING OUT THE INVENTION

Hereinafter, detailed description is given of an embodiment of the present technology with reference to the drawings. The following description is a specific example of the present disclosure, and the present disclosure is not limited to the following embodiment. Further, the present disclosure is not limited to arrangement, dimensions, dimensional ratios, and the like of constituent elements illustrated in the drawings. It is to be noted that the description is given in the following order.
1. Embodiment (an example of a film including a rough surface)
   1-1. Configuration of Film
   1-2. Method of Manufacturing Film
   1-3. Workings and Effects
2. Examples
3. Application Examples

### 1. EMBODIMENT

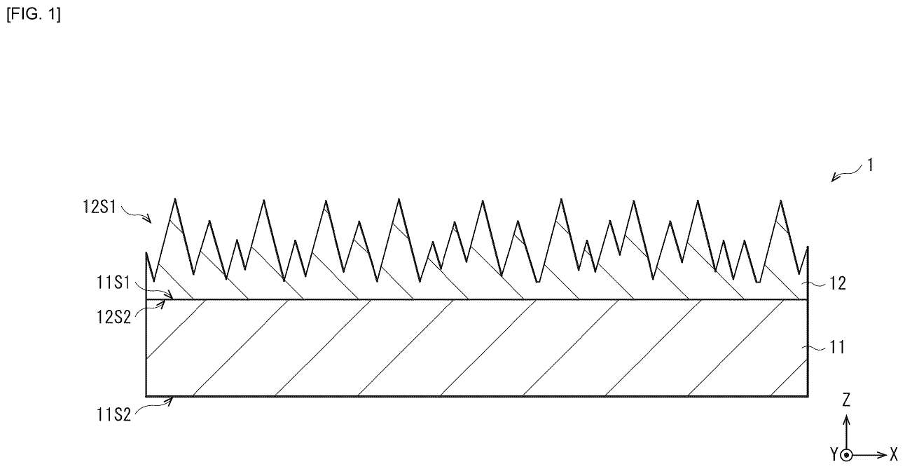

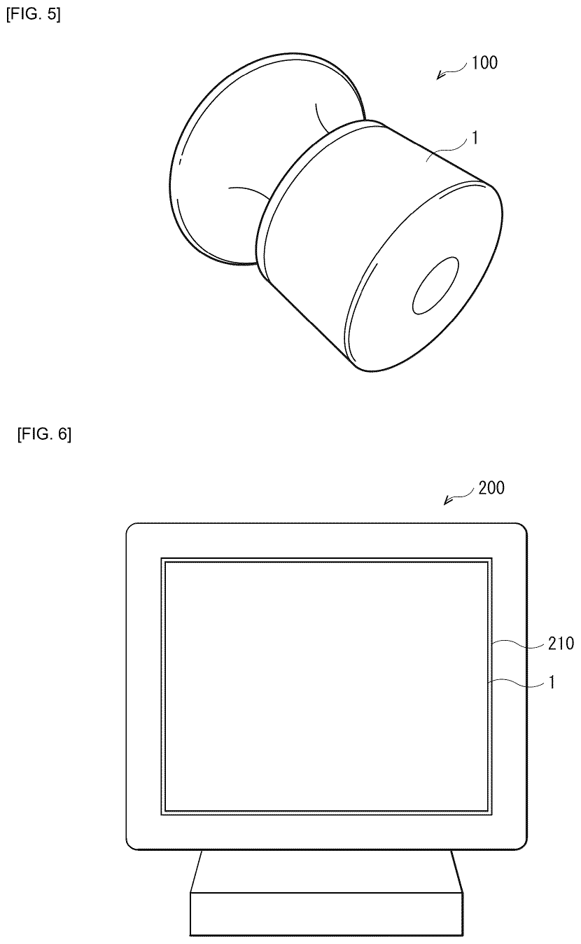

FIG. 1 schematically illustrates a configuration of a film (a film 1) according to an embodiment of the present disclosure. The film 1 is mounted on, for example, a structure such as a doorknob (e.g., a doorknob 100, see FIG. 5) or a display unit of a touch panel display (e.g., a touch panel display 200, see FIG. 6) to be touched by a hand of a person. The film 1 includes a base 11 and a hard coat layer 12. The hard coat layer 12 is formed on one surface of the base 11 and includes a rough surface (a surface 12S1).

### (1-1. Configuration of Film)

The film 1 includes, for example, the base 11 having a pair of opposing surfaces (a surface 11S1 and a surface 11S2) and the hard coat layer 12 having a pair of opposing surfaces (the surface 12S1 and a surface 12S2). The surface 12S1 of the hard coat layer 12 is a rough surface having a random uneven structure, and the opposing surface 12S2 is disposed to oppose one surface (the surface 11S1) of the base 11. The random uneven structure constituting the rough surface (the surface 12S1) of the hard coat layer 12 has an arithmetic mean height (Sa) of 0.09 μm or greater and 1.21 μm or less, and has a ratio (Sq/Sa) between a root mean square height (Sq) and the arithmetic mean height (Sa) of 1.22 or greater and 2.36 or less.

The base 11 corresponds to a specific example of a “base” of the present disclosure and has the pair of opposing surfaces (the surface 11S1 and the surface 11S2). Examples of the base 11 include a plastic film having light transmissivity and flexibility. Examples of the plastic film include plastic films such as polyester films of polyethylene terephthalate, polybutylene terephthalate, or polyethylene naphthalate, polyolefin films including a polyethylene film and a polypropylene film, cellophane, a diacetyl cellulose film, a triacetyl cellulose film, an acetyl cellulose butyrate film, a polyvinylchloride film, a polyvinylidene chloride film, a polyvinyl alcohol film, an ethylene-vinyl acetate copolymer film, a polystyrene film, a polycarbonate film, a polymethylpentene film, a polysulfone film, a polyether ether ketone film, a polyether sulfone film, a polyether imide film, a fluororesin film, a polyamide film, an acrylic resin film, a polyurethane resin film, a norbornene polymer film, a cyclic olefin polymer film, a cyclic conjugated diene polymer film, or a vinyl alicyclic hydrocarbon polymer film, and stacked films thereof.

The base 11 has a thickness of, for example, 4 μm or greater and 250 μm or less, preferably 10 μm or greater and 125 μm or less.

The hard coat layer 12 corresponds to a specific example of a “nanostructure” of the present disclosure, and has, for example, light transmissivity. The hard coat layer 12 has the pair of opposing surfaces (the surface 12S1 and the surface 12S2), and the surface 12S1 is the rough surface having the random uneven structure, as illustrated in FIG. 1. The uneven structure of the surface 12S1 has an arithmetic mean height (Sa) of, for example, 0.09 μm or greater and 1.21 μm or less, more preferably an arithmetic mean height (Sa) of 0.37 μm or greater and 0.89 μm or less. In addition, the uneven structure of the surface 12S1 has a root mean square height (Sq) of, for example, 0.22 or greater and 1.48 or less, and has a ratio (Sq/Sa) between the root mean square height (Sq) and the arithmetic mean height (Sa) of 1.22 or greater and 2.36 or less, more preferably a ratio (Sq/Sa) between the root mean square height (Sq) and the arithmetic mean height (Sa) of 1.24 or greater and 1.53 or less.

It is to be noted that the arithmetic mean height (Sa) is a parameter resulting from expanding Ra (an arithmetic mean height of lines) to a surface, and represents an average of absolute values of differences in heights at respective points with respect to a mean plane of the surface. The root mean square height (Sq) is a parameter corresponding to a standard deviation of a distance from the mean plane and corresponds to the standard deviation of the height.

Examples of the hard coat layer 12 include an organic material such as an acrylic resin, a silicone resin, or an epoxy resin, and an inorganic material such as a silica-containing coating agent.

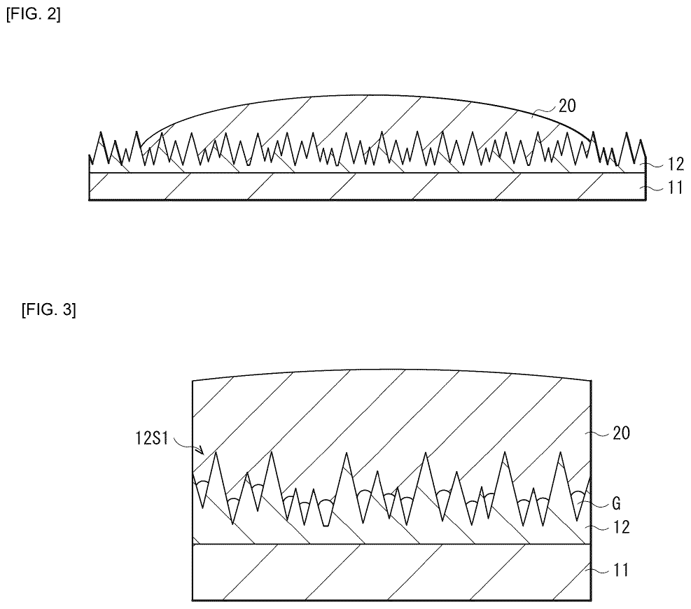

The rough surface (the surface 12S1) of the hard coat layer 12 has lipophilicity. FIG. 2 schematically illustrates a mode obtained when a liquid drop of trioleic acid assumed to be grease of a person is added onto the surface 12S1 of the hard coat layer 12 having the uneven structure. The hard coat layer 12 has higher wettability than a typical hard coat layer because of having a surface area increased by the above-described random uneven structure. Specifically, the surface 12S1 of the hard coat layer 12 has a trioleic acid contact angle of 8.5° or greater and 14.8° or less, more preferably 8.3° or greater and 9.4° or less. It is to be noted that, in a state where a liquid drop 20 of the trioleic acid is allowed to stand still on the surface (the surface 12S1) of the hard coat layer 12, the term “trioleic acid contact angle” refers to an angle that is on a side where the liquid drop is present and is formed between the surface of the hard coat layer 12 and a tangent to the liquid drop at a portion of the liquid drop in contact with the surface of the hard coat layer 12 described above.

It is possible to calculate the contact angle between the trioleic acid and the surface 12S1 of the hard coat layer 12 using a typical method of measuring the contact angle by macroscopically observing the surface 12S1 serving as the rough surface. For example, a drop of liquid is added onto the surface 12S1 of the hard coat layer 12 to observe a shape of the liquid drop from the side. At this time, an angle formed between the surface 12S1 of the hard coat layer 12 and the surface of the liquid is referred to as a contact angle.

The surface 12S1 of the hard coat layer 12 has a wiping property. FIG. 3 schematically illustrates a vicinity of an interface between the trioleic acid and the surface 12S1 of the hard coat layer 12. The trioleic acid has, for example, surface tension (36 mN/m) lower than that of pure water and higher than that of ethanol. The random uneven structure and the surface tension of the trioleic acid described above cause formation of a gap G, into which the liquid drop 20 of the oleic acid is not able to infiltrate, in a clearance space of the uneven structure of the surface 12S1 of the hard coat layer 12. Meanwhile, the uneven structure of the surface 12S1 of the hard coat layer 12 is sized to allow ethanol having surface tension (22 mN/m) lower than that of the trioleic acid to infiltrate into the uneven structure. This allows for easy wiping of the trioleic acid.

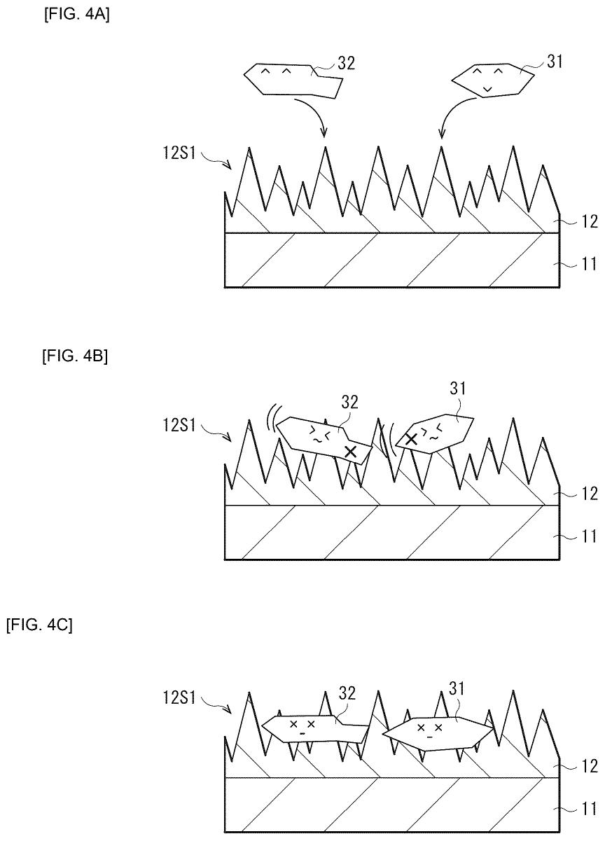

The surface 12S1 of the hard coat layer 12 has antibacterial performance, and, for example, has an antibacterial activity value of 3.0 or greater against Staphylococcus aureus. FIGS. 4A to 4C each illustrate a mechanism of an assumed bactericidal function of the hard coat layer 12. First, for example, protrusions having a height of about several microns in the uneven structure having an arithmetic mean height (Sa) of 0.09 μm or greater and 1.21 μm or less induce attachment of bacteria 31 and bacteria 32, as illustrated in FIG. 4A. A cell wall of each of the bacteria 31 and the bacteria 32 trapped in the protrusions having the height of about several microns is thought to be damaged by protrusions therearound having a sub-micron height to thereby cause water and the like in the cell to flow out, as illustrated in FIG. 4B. This presumably results in death of the bacteria 31 and the bacteria 32 eventually, as illustrated in FIG. 4C. That is, it is presumed that protruding parts having various heights and being present in the uneven structure exhibit a bactericidal effect in a competitive manner.

### (1-2. Method of Manufacturing Film)

It is possible to form the uneven structure of the surface 12S1 of the hard coat layer 12 through, for example, wet blasting. The wet blasting is a surface treatment technique that forms uneven parts on a surface of an object by accelerating water and an abrasive with compressed air and causing collision of the water and the abrasive. For example, the wet blasting enables fine and uniform treatment without generating dust as compared with any other surface treatment technique such as sandblasting. It is possible to control a microstructure formed on the surface of the object by parameters such as a particle size or a type of the abrasive, pressure, velocity, the number of times of treatments, or a working distance (WD).

### (1-3. Workings and Effects)

One surface (the surface 11S1) of the base 11 of the film 1 according to the present embodiment is provided with the hard coat layer 12 having the surface (the surface 12S1) serving as the rough surface that has the random uneven structure and has an arithmetic mean height (Sa) of 0.09 μm or greater and 1.21 μm or less and a ratio (Sq/Sa) between the root mean square height (Sq) and the arithmetic mean height (Sa) of 1.22 or greater and 2.36 or less. This physically traps bacteria. Description is given of this point below.

In recent years, a touch panel including a display device and an input unit has been widely used. In many cases, a surface of the touch panel is provided with a hard coat film having an anti-glare property or a hard coat film that prevents scratches.

These hard coat films are defined to have, for example, an oleic acid contact angle of 45° or greater, to improve a fingerprint wiping property, as described above. In other words, the wiping property is improved by using a material having high oil repellency for the hard coat films. Meanwhile, coating these hard coat films with ink or a resin material causes an issue of poor wettability.

Further, with an increase in hygiene awareness in recent years, it has been demanded to impart antibacterial performance to various members including a building such as a public facility or a commercial facility, a device such as a touch panel, and a general household commodity.

To satisfy this demand, the surface (the surface 12S1) of the hard coat layer 12 of the film 1 according to the present embodiment is the rough surface having the random uneven structure and having an arithmetic mean height (Sa) of 0.09 μm or greater and 1.21 μm or less and a ratio (Sq/Sa) between the root mean square height (Sq) and the arithmetic mean height (Sa) of 1.22 or greater and 2.36 or less. This physically traps and kills bacteria.

The configuration as described above makes it possible to impart durable antibacterial performance to the film 1 according to the present embodiment.

In addition, the film 1 according to the present embodiment is provided with the random uneven structure described above. This allows the film 1 according to the present embodiment to have a larger surface area from a macroscopic viewpoint and thus have improved wettability of, for example, ink or a resin material as compared with a typical hard coat layer. Meanwhile, ink, a resin material, or grease of a person is unlikely to penetrate the gap of the random uneven structure from a microscopic viewpoint, which provides a favorable wiping property. That is, it is possible to achieve both high adhesion and an excellent wiping property.

Further, in the present embodiment, the random uneven structure is provided on the surface (the surface 12S1) of the hard coat layer 12 through wet blasting. This makes it possible to thin the base 11 (e.g., to 4 micrometers), for example. This allows the film 1 to be attached to a curved surface or the like.

It is to be noted that the present embodiment describes the example in which the surface 12S1 of the hard coat layer 12 is the rough surface having the random uneven structure, but the present disclosure is not limited thereto. The effects of the present embodiment are also achievable by forming, for example, the surface 11S1 of the base 11 to be a rough surface having the random uneven structure described above. In this case, it is possible to use, for the base 11, a resin, stone, metal, glass, or a flexible material such as an elastomer in addition to the above-described materials. It is also possible to use, for the base 11, for example, a mold for injection molding subjected to surface treatment by wet blasting.

### 2. EXAMPLES

Table 1 summarizes evaluation results of the arithmetic mean height (Sa), the root mean square height (Sq), the ratio (Sq/Sa) between the root mean square height (Sq) and the arithmetic mean height (Sa), an antibacterial activity value R, a trioleic acid contact angle, an antibacterial effect, lipophilicity, and a wiping property of each of Experimental Examples 1 to 8. In each of Experimental Examples 1 to 8, a polyethylene film is subjected to surface treatment by wet blasting. It is possible to reproduce an uneven structure (the arithmetic mean height (Sa) and the root mean square height (Sq)) of each of Experimental Examples 1 to 8 by appropriately selecting and adjusting an abrasive, air pressure (MPa), and a treatment rate (mm/s). The antibacterial effect, the lipophilicity, and the wiping property were evaluated using the following methods.

### [Method of Evaluating Antibacterial Effect]

The antibacterial effect of each of Experimental Examples 1 to 8 was evaluated by a test method defined in the antibacterial property test method in accordance with JIS Z 2801 (ISO 22196). In the antibacterial property test method, the antibacterial effect is evaluated by the antibacterial activity value obtained using Equation (1) below. In Examples, an antibacterial activity value R of 5 (a saturation value) was defined as A, an antibacterial activity value R of 3.0 or greater and less than 5.0 was defined as B, and an antibacterial activity value R of less than 3.0 was defined as C.

$$\begin{matrix}( Math . 1 ) & \\ R = ( U t - {U}_{0} ) - ( A t - {U}_{0} ) = U t - A t & ( 1 )\end{matrix}$$

(R denotes an antibacterial activity value, U₀ denotes an average logarithmic value of viable bacteria values immediately after inoculation of an untreated specimen (a reference), Ut denotes an average logarithmic value of viable bacteria values 24 hours after the inoculation of the untreated specimen, and At denotes an average logarithmic value of viable bacteria values 24 hours after inoculation of a specimen subjected to an antibacterial treatment.)

### [Evaluation of Lipophilicity]

A trioleic acid contact angle of 15.0° or less was defined as A and a trioleic acid contact angle of greater than 15.0° was defined as B.

### [Evaluation of Wiping Property]

A wiping test of trioleic acid was conducted assuming that the trioleic acid is grease of a fingertip of a person. After a drop of the trioleic acid was added onto the polyethylene film of each of Experimental Examples 1 to 8, a USL type of a swab for a clean room (manufactured by SANSYO Co., LTD.) sufficiently immersed in ethanol (purity: 99.5%) was used to perform a wiping operation in a vertical direction with a weight of the fingertip of about 500 gf. When the wiping operation was performed for five reciprocations and ten reciprocations, the polyethylene film was dried and an image of a surface of the polyethylene film was captured and visually judged. In Examples, when the wiping operation had been performed for ten reciprocations, a case where the trioleic acid was completely wiped was defined as A, a case where the trioleic acid was left without being partly wiped was defined as B, and a case where the trioleic acid was spread and became entirely cloudy was defined as C.

TABLE 1

| | | | | | Trioleic | | | |
|---|---|---|---|---|---|---|---|---|
| | | | | Antibacterial | Acid | | | |
| | Sa | Sq | | Activity | Contact | Antibacterial | | Wiping |
| Index | (μm) | (μm) | Sq/Sa | Value R | Angle | Effect | Lipophilicity | Property |
| Experimental | 0.01 | 0.01 | 1.33 | 2.8 | 16.3 | C | B | C |
| Example 1 | | | | | | | | |
| Experimental | 0.05 | 0.09 | 1.96 | 2.8 | 15.2 | C | B | B |
| Example 2 | | | | | | | | |
| Experimental | 0.09 | 0.22 | 2.36 | 3.7 | 14.8 | B | A | A |
| Example 3 | | | | | | | | |
| Experimental | 0.37 | 0.56 | 1.53 | 5.0 | 9.4 | A | A | A |
| Example 4 | | | | | | | | |
| Experimental | 0.68 | 0.87 | 1.28 | 5.0 | 8.7 | A | A | A |
| Example 5 | | | | | | | | |
| Experimental | 0.89 | 1.10 | 1.24 | 5.0 | 8.3 | A | A | A |
| Example 6 | | | | | | | | |
| Experimental | 1.21 | 1.48 | 1.22 | 3.6 | 8.5 | B | A | A |
| Example 7 | | | | | | | | |
| Experimental | 1.50 | 1.92 | 1.28 | 2.2 | 7.9 | C | A | B |
| Example 8 | | | | | | | | |

In view of Table 1, a sufficient antibacterial effect, lipophilicity, and wiping property were obtained in each of Experimental Examples 3 to 7 having an arithmetic mean height (Sa) of 0.09 μm or greater and 1.21 μm or less and a ratio (Sq/Sa) between the root mean square height (Sq) and the arithmetic mean height (Sa) of 1.22 or greater and 2.36 or less. It has been particularly found that a higher antibacterial effect was obtained in each of Experimental Examples 4 to 6 having an arithmetic mean height (Sa) of 0.37 μm or greater and 0.89 μm or less and a ratio (Sq/Sa) between the root mean square height (Sq) and the arithmetic mean height (Sa) of 1.24 or greater and 1.53 or less.

### 3. APPLICATION EXAMPLES

The film 1 according to the above-described embodiment is attachable to the doorknob 100 illustrated in FIG. 5 or a display unit 210 of the touch panel display 200, for example.

In addition thereto, the film 1 is usable at various locations to be touched by a person, such as a floor or a wall of a room, a desk, or a hanging strap on a train. Further, the film 1 is usable for any surfaces of products that are likely to be touched by a person, such as a food packaging film, a surface of a beverage can, a vending machine, various switches, a controller, a remote control, a smartphone, a PC, stationery, a vehicle, an in-vehicle device, water facilities including a bathroom, a washbasin, and a pool, or various optical products. Furthermore, the film 1 is also usable for any surfaces of products that are unlikely to be touched by a person, such as a building material for a ceiling, a pillar, or an outer wall of a house, an anti-glare film or a diffusion film of a monitor, a polarizing plate, surfaces of various sensing members, or a refrigerator.

Although the present disclosure has been described with reference to the embodiment and the modification examples, the present disclosure is not limited to the above-described embodiment and the like, and various modifications are possible.

It is to be noted that the effects described herein are merely exemplary and the effects of the present disclosure are not limited to the effects described herein. The present disclosure may have any other effects than the effects described herein.

It is to be noted that the present technology may also have the following configurations. According to the present technology having the following configurations, one surface (a first surface) of a pair of opposing surfaces is provided with a random uneven structure having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less. Such a configuration achieves durable antibacterial performance of physically trapping and killing bacteria.
(1)
   A nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.
(2)
   The nanostructure according to (1), in which
   the arithmetic mean height (Sa) of the rough surface is 0.37 μm or greater and 0.89 μm or less, and
   the ratio (Sq/Sa) between the root mean square height (Sq) and the arithmetic mean height (Sa) of the rough surface is 1.24 or greater and 1.53 or less.
(3)
   The nanostructure according to (1) or (2), in which the rough surface has a trioleic acid contact angle of 8.5 degrees or greater and 14.8 degrees or less.
(4)
   The nanostructure according to (1) or (2), in which the rough surface has a trioleic acid contact angle of 8.3 degrees or greater and 9.4 degrees or less.
(5)
   The nanostructure according to any one of (1) to (4), in which the rough surface has a wiping property.
(6)
   The nanostructure according to any one of (1) to (5), in which the rough surface has an antibacterial activity value of 3.0 or greater against Staphylococcus aureus.
(7)
   The nanostructure according to any one of (1) to (6), in which the nanostructure has light transmissivity.
(8)
   A method of manufacturing a nanostructure, the method including forming a random uneven surface in the nanostructure through wet blasting, the random uneven surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.
(9)
   A film including a layer including a nanostructure, the nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.
(10)
   A structure including:
   a base; and
   a film including a layer including a nanostructure, the nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

The present application claims the benefit of Japanese Priority Patent Application JP2022-129643 filed with the Japan Patent Office on Aug. 16, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. A nanostructure comprising a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

2. The nanostructure according to claim 1, wherein
the arithmetic mean height (Sa) of the rough surface is 0.37 μm or greater and 0.89 μm or less, and
the ratio (Sq/Sa) between the root mean square height (Sq) and the arithmetic mean height (Sa) of the rough surface is 1.24 or greater and 1.53 or less.

3. The nanostructure according to claim 1, wherein the rough surface has a trioleic acid contact angle of 8.5 degrees or greater and 14.8 degrees or less.

4. The nanostructure according to claim 1, wherein the rough surface has a trioleic acid contact angle of 8.3 degrees or greater and 9.4 degrees or less.

5. The nanostructure according to claim 1, wherein the rough surface has a wiping property.

6. The nanostructure according to claim 1, wherein the rough surface has an antibacterial activity value of 3.0 or greater against Staphylococcus aureus.

7. The nanostructure according to claim 1, wherein the nanostructure has light transmissivity.

8. A method of manufacturing a nanostructure, the method comprising forming a random uneven surface in the nanostructure through wet blasting, the random uneven surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

9. A film comprising a layer including a nanostructure, the nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.

10. A structure comprising:
a base; and
a film including a layer including a nanostructure, the nanostructure including a rough surface, the rough surface having an arithmetic mean height (Sa) of 0.09 μm or greater and 1.21 μm or less and a ratio (Sq/Sa) between a root mean square height (Sq) and the arithmetic mean height (Sa) of 1.22 or greater and 2.36 or less.
